(19) United States
(12) Patent Application Publication
(10) Pub. No.: US 2013/0218214 A1
(43) Pub. Date: Aug. 22, 2013

(54) BONE SCREW HEAD DESIGN

(71) Applicant: Carbofix Orthopedics Ltd., (US)

(72) Inventors: Mordechay BEYAR, Caesarea (IL); Oren GLOBERMAN, Kfar-Shemaryahu (IL)

(73) Assignee: Carbofix Orthopedics Ltd., Herzlia Pituach (IL)

(21) Appl. No.: 13/852,145

(22) Filed: Mar. 28, 2013

Related U.S. Application Data
(63) Continuation-in-part of application No. 13/742,462, filed on Jan. 16, 2013.
(60) Provisional application No. 61/617,067, filed on Mar. 29, 2012.
(60) Provisional application No. 61/641,900, filed on May 3, 2012.
(60) Provisional application No. 61/586,853, filed on Jan. 16, 2012.
(60) Provisional application No. 61/617,067, filed on Mar. 29, 2012.
(60) Provisional application No. 61/641,900, filed on May 3, 2012.

(51) Int. Cl. A61B 17/86

(52) U.S. Cl. CPC A61B 17/862
USPC 606/305

(57) ABSTRACT

A screw head design, usable for composite material bone screws, in which compression forces applied to the screw head are reduced by one or more of location, size and/or shape of screw head design. In some embodiments, load bearing recesses are located closer to an outer circumference of a screw head than to its central axis.

### RELATED APPLICATIONS

This application claims the benefit of priority under 35 USC §119(e) of U.S. Provisional Patent Application Nos. 61/617,067 filed Mar. 29, 2012 and 61/641,900 filed May 3, 2012.

This application is also a Continuation-in-Part (CIP) of U.S. patent application Ser. No. 13/742,462 filed Jan. 16, 2013, which claims the benefit of priority under 35 USC §119(e) of U.S. Provisional Patent Application Nos. 61/586,853 filed Jan. 16, 2012, 61/617,067 filed Mar. 29, 2012 and 61/641,900 filed May 3, 2012.

This application is also related to a co-filed U.S. Continuation-in-Part (CIP) Patent Application titled “Bone Screw With Insert” and having attorney docket number 56167.

This application is also related to PCT Patent Application Nos. PCT/IB2011/052468 filed on Jun. 7, 2011 and PCT/IB2010/050225 filed on Jan. 18, 2010.

The contents of the above applications are all incorporated by reference as if fully set forth herein in their entirety.

### FIELD AND BACKGROUND OF THE INVENTION

The present invention, in some embodiments thereof, relates to screw head designs for bone screws, more particularly, but not exclusively, to designs for a screw formed of polymer and/or composite materials.

Bone screws are well known in the art and are used, for example, to interconnect bone and to attach implants to bones. A typical bone screw includes a head to which a screw driver is couplable, a body, generally cylindrical or conical and/or including a tapering tip and one or more threads on the body. Various head designs have been proposed for bone screws, including, for example, axial crown, slotted, hexalobe, internally or externally threaded hexagon and Phillips type heads.

Additional background art includes US Patent Application Publication No. 2003/57590.

### SUMMARY OF THE INVENTION

There is provided in accordance with an exemplary embodiment of the invention an orthopedic screw formed at least in part of a composite or polymer material, comprising:

(a) a generally cylindrical body defining an axis and including at least a partial threading thereon;

(b) a tip at a distal side of the body;

(c) a head at a proximal side of the body, said head having a maximal radius and defining at least one receptacle in the form of a recess, for a blade of a screwdriver,

wherein said recess includes a load bearing section adapted to engage said blade and which is closer to a circumference of said screw head than to an axis of said body.

In an exemplary embodiment of the invention, said screw is at least 90% by volume formed of a composite material or polymer. Optionally or alternatively, said load bearing section is within 40% of said maximal radius from a circumference of said screw head. Optionally, said load bearing section is within 30% of said maximal radius from a circumference of said screw head. Optionally, all of the load bearing sections of said screw head are at least 20% of said maximal radius distanced from said axis.

In an exemplary embodiment of the invention, at least 50% of the load bearing sections of said screw head are at least 40% of said maximal radius distanced from said axis.

In an exemplary embodiment of the invention, the screw comprises a plurality of recesses which are not connected. Optionally, the screw comprises at least 3 unconnected recesses. Optionally or alternatively, a shape of said recesses is substantially circular. Optionally or alternatively, at least one of said recesses extends more in a circumferential direction than a radial direction of said head. Optionally or alternatively, said recesses are surrounded on all sides by a surface of said head.

In an exemplary embodiment of the invention, said recess forms an edge of said screw head.

In an exemplary embodiment of the invention, said recess is in the form of a slot.

In an exemplary embodiment of the invention, said recess is at least 1 mm deep at its most shallow portion.

In an exemplary embodiment of the invention, said recess has a varying width as a function of depth.

In an exemplary embodiment of the invention, said recess has associated therewith at least one insert formed of a material harder than said composite material or polymer, located at a load bearing surface thereof which is designed to receive force from said blade when said screw is driven.

In an exemplary embodiment of the invention, the screw comprises at least one screwdriver guiding geometry formed in said head. Optionally, said geometry comprises a protrusion from a surface of said screw head. Optionally or alternatively, said geometry comprises a depression in a surface of said screw head. Optionally or alternatively, geometry is rotationally symmetric with respect to said screw axis.

In an exemplary embodiment of the invention, the screw comprises at least one orientation guide formed along a circumference of said screw head.

In an exemplary embodiment of the invention, at least said head is formed of a composite material including as tensile elements short chopped fibers.

In an exemplary embodiment of the invention, at least said head is formed of a composite material having a better compression resistance than said body of said screw.

In an exemplary embodiment of the invention, said screw is in the shape of a lag screw.

In an exemplary embodiment of the invention, said screw is in the shape of a self tapping screw. Optionally, said screw tip is configured to drill into bone.

In an exemplary embodiment of the invention, the screw is provided in kit form with a matching screwdriver having a blade adapted to frictionally engage said at least one receptacle.

In an exemplary embodiment of the invention, the screw is provided in kit form with a matching screwdriver having a blade adapted to engage said at least one receptacle. Optionally, said kit comprises a plurality of bone screws. Optionally or alternatively, said kit comprises one of a bone nail and a bone plate.

There is provided in accordance with an exemplary embodiment of the invention a screwdriver for a composite orthopedic screw comprising a shaft and a to blade section, wherein said blade section defines one or both of an axial hollow extending to a distal tip thereof and axially extending guide which is rotationally symmetric.

There is provided in accordance with an exemplary embodiment of the invention a kit comprising a screwdriver having a blade and an orthopedic screw having a receptacle for said blade, wherein a geometry of said blade interferes with a geometry of said receptacle, so as to provide friction engagement of said blade by said screw, when said blade is inserted into said receptacle.

There is provided in accordance with an exemplary embodiment of the invention an orthopedic screw having a generally cylindrical body having an axis and a thread on said body, wherein said body comprises a cylindrical section with a diameter of at least 50% of a diameter of said body, in which all elongate fibers are substantially aligned with said axis.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

Implementation of the method and/or system of embodiments of the invention (e.g., control of manufacturing methods) can involve performing or completing selected tasks manually, automatically, or a combination thereof. Moreover, according to actual instrumentation and equipment of embodiments of the method and/or system of the invention, several selected tasks could be implemented by hardware, by software or by firmware or by a combination thereof using an operating system.

For example, hardware for performing selected tasks according to embodiments of the invention could be implemented as a chip or a circuit. As software, selected tasks according to embodiments of the invention could be implemented as a plurality of software instructions being executed by a computer using any suitable operating system. In an exemplary embodiment of the invention, one or more tasks according to exemplary embodiments of method and/or system as described herein are performed by a data processor, such as a computing platform for executing a plurality of instructions. Optionally, the data processor includes a volatile memory for storing instructions and/or data and/or a non-volatile storage, for example, a magnetic hard-disk and/or removable media, for storing instructions and/or data. Optionally, a network connection is provided as well. A display and/or a user input device such as a keyboard or mouse are optionally provided as well.

### BRIEF DESCRIPTION OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIGS. 1A-1E illustrate a geometry for connecting a screw head to a screw driver in accordance with some exemplary embodiments of the invention;

FIGS. 2A-2D show an alternative geometry, in accordance with exemplary embodiments of the invention;

FIG. 2E is a top view of a screw head illustrating various geometric considerations in accordance with exemplary embodiments of the invention;

FIGS. 3A-3D show a further alternative geometry, in accordance with exemplary embodiments of the invention;

FIGS. 4A-4C show a further alternative geometry, in accordance with exemplary embodiments of the invention;

FIG. 5 shows a screwdriver geometry matching the geometry of a screw head shown in FIGS. 4A-4C, in accordance with exemplary embodiments of the invention;

FIGS. 6A-6C schematically illustrate a composite material screw and design, in accordance with some embodiments of the invention.

FIGS. 7A-F schematically illustrate manufacturing methods of composite material bone screw as in prior art and in accordance with some embodiments of the invention; and

FIG. 8 schematically illustrates a cannulated composite material bone screw comprising a head made of different material, in accordance with some embodiments of the invention.

### DESCRIPTION OF SPECIFIC EMBODIMENTS OF THE INVENTION

The present invention, in some embodiments thereof, relates to screw head designs for bone screws, more particularly, but not exclusively, to designs for a screw formed of polymer and/or composite materials.

### Overview

An aspect of some embodiments of the invention relates to a screw head design, optionally for bone screws, in which compression and/or other forces on the screw head, during rotation thereof, are reduced. In an exemplary embodiment of the invention, the bone screw head is formed of a composite and/or polymer material, which may be otherwise more prone to damage during typical use of the screw.

In an exemplary embodiment of the invention, the geometry of the connection means (i.e., the interface between a composite material screw head and a metal (or other material screwdriver) is such that reduces the compression pressure applied on the screw head for threading and/or unthreading the screw, potentially reducing the potential for screw head damage.

In an exemplary embodiment of the invention, the interface is not located at the center of the screw head but rather more peripherally. Optionally, said interface is not at the screw head circumference, to provide for smooth circumference surface and potentially prevent potential harm to soft tissue adjacent the screw head.

In an exemplary embodiment of the invention, the force applied by the screwdriver is on a vector perpendicular to radial. In an embodiment, the geometry of said connection means comprises one or more recesses, having a shape such as square, circle, ellipse, banana-shape, or other shape.

In some embodiments, the screwdriver comprises complementary geometry to match the geometry of the screw head. In some embodiments, the geometry of the screw head is such that facilitates screwdriver precise engagement with the screw head for engaging the recess(es).

In an embodiment, the interface geometry is designed to provide self-retaining of the screw head by the screwdriver, optionally due to distortion of the composite material by the screwdriver in a way that engages the screwdriver. Optionally, self retaining is provided using tapering of one or both of the screwdriver and one or more of the recesses.

In an exemplary embodiment of the invention, two or more recesses are defined in the screw head to receive a screw-driving instrument. In an exemplary embodiment of the invention, the recesses include significant load bearing portions which transfer load from the screwdriver during driving of the screw and which are located at a significant distance from the longitudinal axis of the screw, for example, further than the radius of the screw body, further than half the outer radius of the head or further. Optionally, all of the load bearing portion which bear circumferentially directed load are at least 30%, 50%, 60%, 70, 80% or intermediate percentages of the head radius, from the screw axis.

It should be noted that in many uses, it may be desirable to reduce the mechanical gain provided by the relative location of the load bearing portions and the screw axis, so as to make it less likely that the head have too much torque and/or local compression stress applied to it and possibly tearing the screw head off of the screw body.

In an exemplary embodiment of the invention, however, an issue to be addressed is preventing of damage to the screw head itself. Locating the load bearing portions closer to the screw axis increases the force applied to the head in a circumferential direction and in sensitive materials may cause failure, such as due to crushing, shearing or splitting.

In an exemplary embodiment of the invention, the load bearing portions extend a significant amount in a circumferential direction, optionally at the expense of extension in a radial direction (e.g., of the screw head). In an exemplary embodiment of the invention, the extension length in the circumferential direction is between 80% to and 600% of the extension in the radial direction.

In some embodiments of the invention the load bearing section also extends radially towards the head center, closer than half the head maximal radius. This may be useful, for example, for guiding the screwdriver into the load bearing portions.

In an exemplary embodiment of the invention, the width of the load bearing section (in a circumferential direction) is greater closer to the screw axis. Optionally or alternatively, the screwdriver blades are narrower at the portion designed to engage the load bearing sections nearer the screw head center. This may allow the load bearing section to act as a guide for the screw driver, while reducing excess strain at low radial distances.

In an exemplary embodiment of the invention, an insert, for example, of metal, or other material more resistant to shear forces and/or damage by compression forces and/or other forces associated with screwdriving, is placed in the recesses to mechanically couple the screw driving force to the screw head. In an exemplary embodiment of the invention, such an insert has a thickness of between 0.01 mm and 2 mm.

In an exemplary embodiment of the invention, the slots are at least 0.1 mm, 0.5 mm, 1 mm deep or intermediate depths, optionally at least 50%, 80%, 100%, 200% or greater or intermediate percentages of the minimal extent of depth in the screw head surface. Optionally, this allows a greater area of contact and thus reduced pressure and risk of damage on the load bearing recesses. Optionally, one or more of the recesses narrows towards its bottom, for example, to ensure engaging of the screwdriver and/or to reduce interaction of the screwdriver with surface parts of the screw head. Optionally, the depth increases as a function of distance from the screw axis.

In an exemplary embodiment of the invention, cruciform slots are used (e.g., similar to a Phillips head design), however, the slots do not become substantially more shallow (e.g., remain at least 20% of maximum depth) away from the screw axis. This is one example of a design which applies screw rotation forces over a long (e.g., >30%, 50%, 70%, 80% of head radius) and in a direction generally. This may result in transferring maximal moment during application of minimal local compressing.

An aspect of some embodiments of the invention relates to screwdriver engaging load bearing recesses which extend in a circumferential direction at least 80% of their extent in a radial direction. Optionally, the extension is at least 100%, 140%, 200%, 300% or intermediate or greater percentages.

In some embodiments the recesses are circular. In some embodiments the recesses are arcuate shaped. In some embodiments the recesses comprises cut-outs at the edge of the screw head.

An aspect of some embodiments of the invention relates to a screw head design which better resists damage by a screwdriver driving force. In an exemplary embodiment of the invention, the screw head includes one or more inserts, for example, to prevent wear of screw and/or redistribute forces applied by the screwdriver, for example, circumferential forces. Optionally or alternatively, the screw head is formed of a composite material and uses unordered chopped fibers and/or other composition which has better compression behavior at the expense of tensile behavior. Optionally or alternatively, the screw head is covered by and/or is formed of a hard substance such as ceramics or metal, mounted on a softer screw body. Optionally or alternatively, the screw head receptacles for the screwdriver are designed to engage the screwdriver away from the surface of the head (e.g., deeper than, for example, 0.1, 0.5 mm or intermediate distances).

An aspect of some embodiments of the invention relates to a screw head design including one or more protrusions and/or depressions designed to guide a screwdriver. In an exemplary embodiment of the invention, the depression and/or protrusion are round, so the screwdriver can first be axially aligned and then rotated until it engages load bearing receptacles. Optionally or alternatively, this prevents shearing of such a protrusion by the screwdriver. In an alternative design, the screw head includes one or more alignment protrusion and/or recess along its circumference. Optionally, such alignment element is easily visible and/or is marked by color or finish.

An aspect of some embodiments of the invention relates to a screwdriver suitable for engaging screws as described above. For example, the screwdriver may have one or more extensions which match said load bearing portions. Optionally or alternatively, the screwdriver includes one or more recesses and/or projections which to match a corresponding part of the screw, for alignment. Optionally or alternatively, the screwdriver is selected to have a somewhat mismatched geometry so as to ensure a friction engaging of the screw by the screwdriver.

In an exemplary embodiment of the invention, the composite material bone screw comprises a head made of metal, such as titanium alloy. Such screw head, constructed from material having a relative high resistance to compression (e.g., as compared to composite material), may be connected to the composite material screw, for example, by compression molding, by geometric connection, adhesion, mechanical connection and/or by other methods, such as known in the art.

In an exemplary embodiment of the invention, the composite material screw, including its thread, is manufactured from fiber-reinforced polymer using compression molding process. In an embodiment, most of the core of the screw comprises straight elongated reinforcing filaments, while the thread teeth and/or the outer portion of the core of the screw comprise elongated filaments which are axially pressed to gain the shape of the thread at the mold circumference (i.e., filaments with a wave-like shape). In an embodiment, a method of manufacturing such a screw comprises compression molding of a composite material rod while applying restraining means to the core elements during the process. In an embodiment, the core fiber elements at least at one of the rod ends are kept straight (e.g., by tension) outside of the mold, optionally in a cold environment, while the circumference fiber elements are axially pressed, optionally by using a cylindrical shape press, so that they are forced to enter into the teeth-shape parts of the mold.

While this is described for forming a screw, a similar method may be used for forming other composite material devices, such as bone implants, using compression molding technique, in which the direction and/or shape of some of the reinforcing fibers is selectively controlled using restraining means during the molding process and/or by compressing other fibers. This can results in devices with desired configuration and preferred mechanical properties, including devices with various different configurations and mechanical properties for different components/portions.

In some cases, the screw having a core with straight longitudinal fibers and a thread with folded longitudinal fibers provides a significant improvement on the to bending strength of a screw compared to a screw constructed only from folded longitudinal fibers.

An aspect of some embodiments of the invention relates to a composite bone screw having an elongate core formed of at least 90% straight fibers, for example, where fibers which do not deviate over more than 10% of their length from a corridor of 1 mm diameter. In some embodiments, 90% or 80% or 70% at least of the fibers have a bending radius of at least 10 mm, 20 mm, 30 mm or more. Optionally, bending at an end of the fibers (e.g., for a 180 degree bend) is allowed.

In an exemplary embodiment of the invention, the straight fiber elongate core extends over between 50% and 90% or more of the diameter of the screw body, for at least 60%, 70%, 80% or more of the screw body length.

Optionally, wavy fibers extend over at least 80% of a depth of the threads and/or over between 10% and 30% of a diameter of the screw body. In an exemplary embodiment of the invention, the degree of waviness increases as a linear increasing function of distance from the screw axis.

Before explaining at least one embodiment of the invention in detail, it is to be understood that the invention is not necessarily limited in its application to the details of construction and the arrangement of the components and/or methods set forth in the following description and/or illustrated in the drawings and/or the Examples. The invention is capable of other embodiments or of being practiced or carried out in various ways.

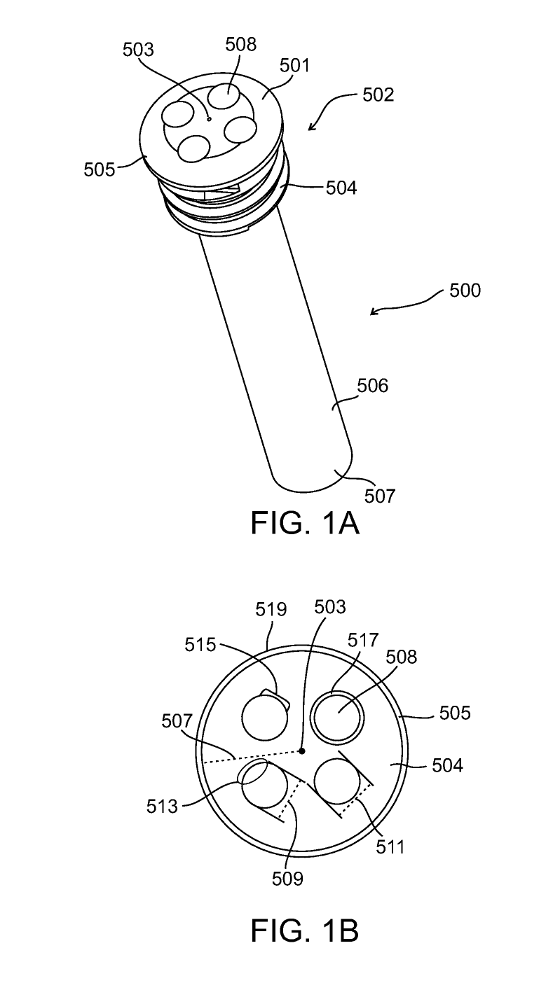

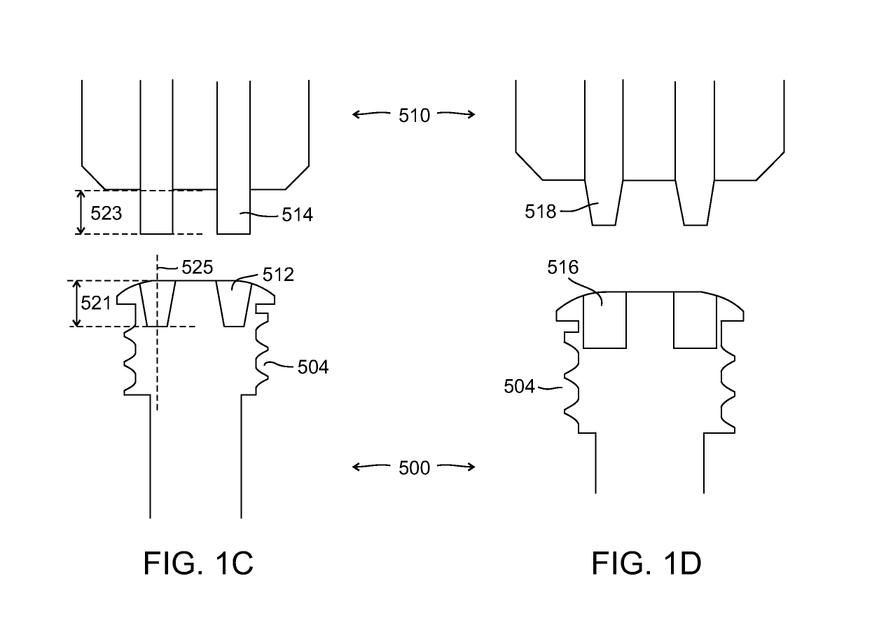

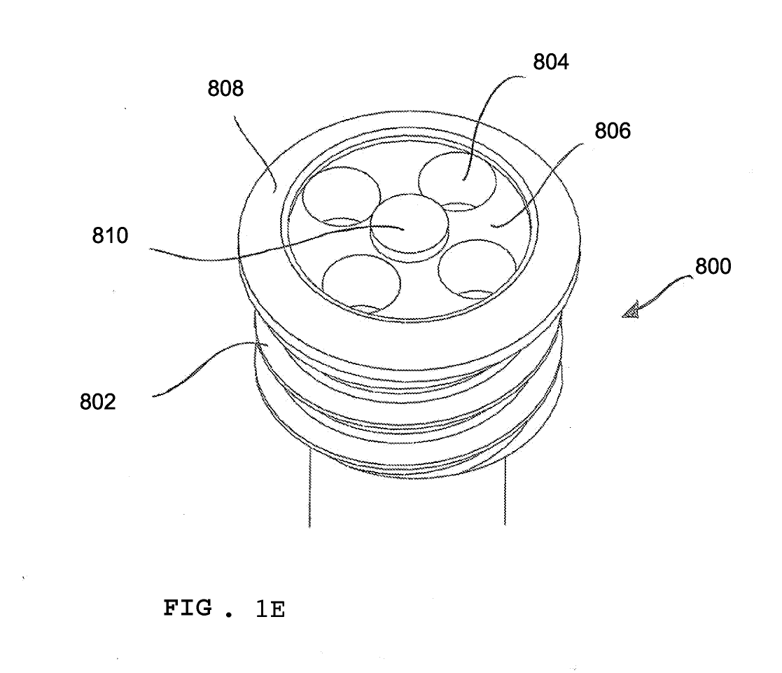

Referring now to the drawings, FIGS. 1-3 illustrate various designs for geometries of a screw head and a screwdriver, in accordance with exemplary embodiments of the invention.

A potential advantage of these geometries is that the engagement of the screw head by the screwdriver is closer to the periphery of the screw head, potentially reducing the forces applied to the screw head and therefore potentially preventing damage to the head.

### Exemplary Screw Head Geometry

FIG. 1A schematically illustrates a composite material bone screw 500, which may be implanted in a bone, for example in conjunction with bone plate or intramedullary nail.

In the illustrated embodiment, screw 500 comprises a head 502, a body (shank) 506 and a tip 507. Optionally, head 502 includes a thread 504, for example, for locking to a bone plate. In the illustrated embodiment, body 506 is smooth. In alternative embodiments, body 506 is partially or completely threaded. Optionally or alternatively, tip 507 may be pointed and/or designed for bone penetration. Optionally or alternatively, screw 500 is a self-tapping bone screw.

Referring to screw head 502, which is shown from a top view in FIG. 1B, a plurality of receptacles 508 are defined for and act as means for engaging and receiving blades of a screwdriver (shown in FIG. 1C). While four recesses are shown, a larger or lesser number may be used, for example, 2 or 3. Optionally, the number of recesses matches the number of blades of the screwdriver, but may also be greater. In an exemplary embodiment of the invention, the recesses are arranged on head 502 in a rotationally symmetric manner. In some embodiments, the arrangement is not symmetrical, or at least limits the number of allowed orientations of the screwdriver to the screw head, for example, based on differences in size, shape and/or positions of the receptacles 508.

Referring to 501 as the surface of head 502 and 503 as the intersection of surface 501 with a main axis of screw 500 (generally the center of head 502) and also referring to a periphery 505 of head 502, the following observations may be made.

In an exemplary embodiment of the invention, the recesses 508 are not located at the center of the screw head 504, but more peripherally. Reference 507 shows a (maximal) radius of head 502. And it can be seen that at least part of a receptacle 508 extends past its mid point. In other embodiments other percentages (e.g., 20%, 30%, 50%, 70%, 90% or intermediate or greater percentages) of the receptacle (as measured by projection of the receptacle on radius 507) extend past other points on the maximal radius (e.g., 20%, 30%, 50%, 60% of the radius and/or intermediate or greater percentages thereof). In particular, a load bearing region 513 is marked on the figure which may be especially prone to damage and part or all of which may be located, for example, past the midpoint of radius 507.

Such design is not commonly used in metal screws, where the standard to connection means is normally located at or near the center of the screw head. A potential advantage of a connection means with peripheral geometry for composite materials, is permitting the application of lower pressures upon rotating the screw with a screwdriver. Compared to metals, composite materials are less generally resistant to compression and/or shear, and may be more prone to damage upon torque application as in screwing a screw. Pressure and/or stress are optionally reduced by one or more of peripheral location, larger and/or more uniform (e.g., with respect to contact quality) contact areas and/or control of angle of force application.

Also shown in FIG. 1B are measurements 509, of the radial extent of a receptacle 508 and 511, a measure of the circumferential extent of a receptacle 508.

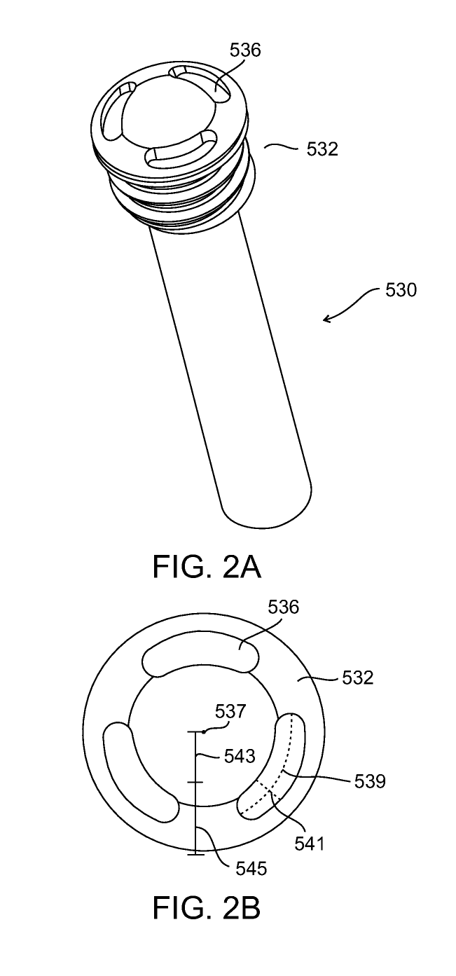

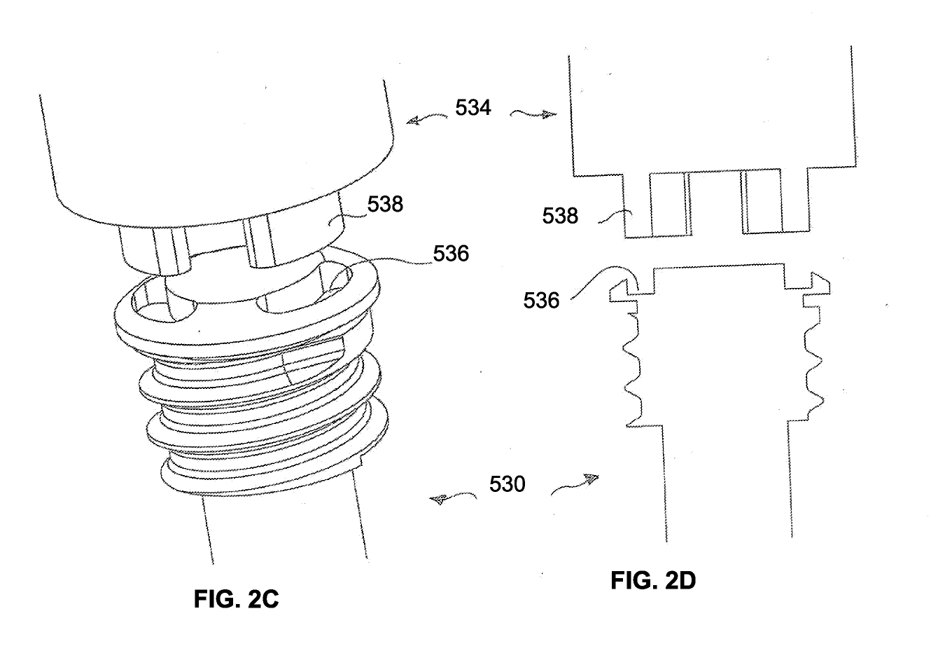

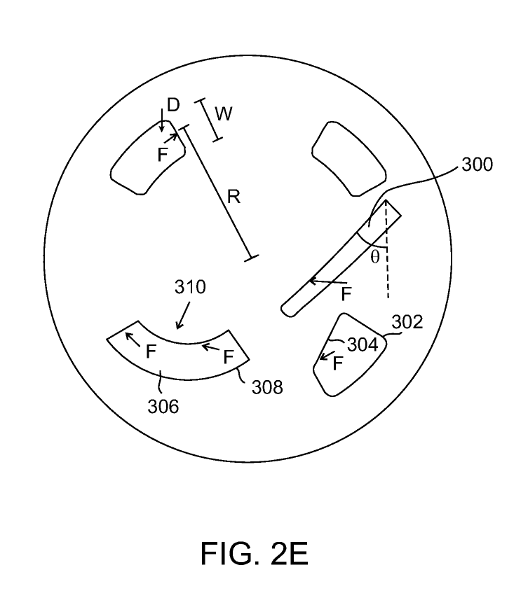

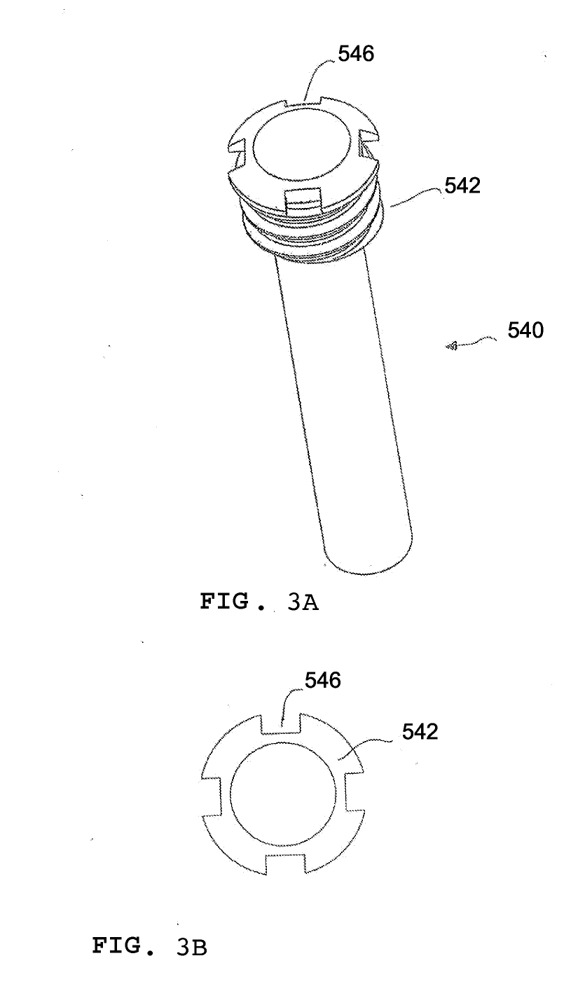

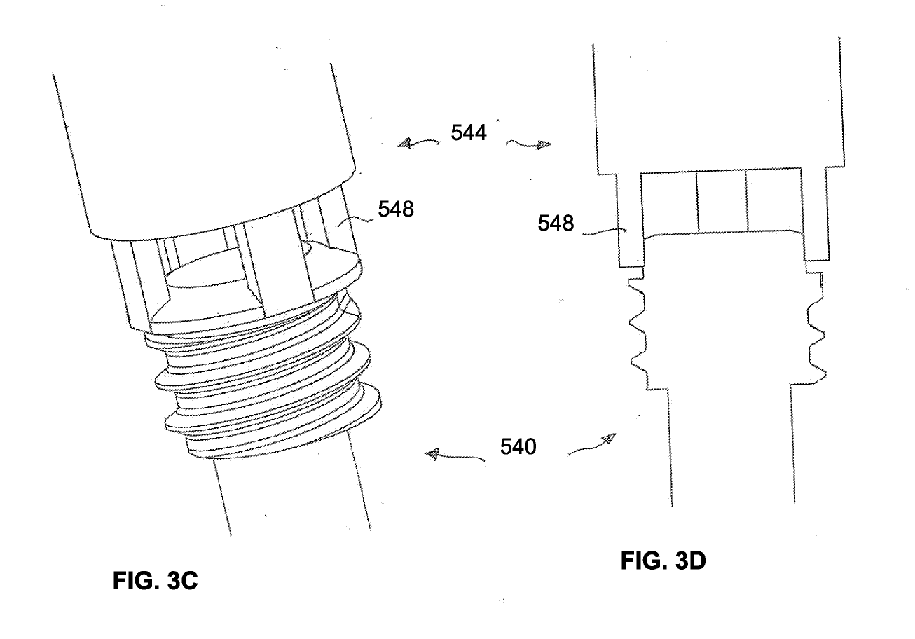

As shown, receptacles 508 have a round shape. Some alternative shapes, numbers and/or locations for recesses are shown in FIGS. 2-3. While generally one or more of the perfusions may be replaced by a protrusion (to match a recess in the screwdriver), in an exemplary embodiment of the invention, this is avoided. Using recesses may allow a lower profile screw head to be used and/or may reduce stress in the screw head, for example possibly avoid failure due to shear forces.

In an exemplary embodiment of the invention, one or more inserts of harder material are used to protect screw head 502. In one example, a ring 519 is provided surrounding part or all of periphery 505 of screw head 502. In another example, an optionally ring shaped insert 517 is provided lining the inside of a receptacle 508. In another example, an insert 515 is provided to protect only a load bearing section of a receptacle 508 and/or only during one of screwing and unscrewing.

### Exemplary Screwdriver and Engagement/Retention

FIGS. 1C and 1D are longitudinal cross sections of the proximal portion of the screw 500 shown in FIG. 1A, and the distal portion of a screwdriver 510, in accordance with exemplary embodiments of the invention.

Referring specifically to FIG. 1C, one or more blades 514 of screwdriver 510 fit into receptacles 512 of screw 500. While In an exemplary embodiment of the invention, blades 514 match receptacles 512, in some embodiments of the invention some mismatch is provided, for example, in a radial and/or circumferential direction, and/or as illustrated here, in an axial direction. Such mismatch may provide for better engagement of the screw by the screwdriver and/or prevent the screw from slipping out of the screwdriver.

This may also allow such retention in case of avoidance of axial pressure on the screw. Optionally, the amount of retention matches or exceeds that provided by magnetic heads and steel screws. In an exemplary embodiment of the invention, the strength of coupling is capable of resisting a force of, for example, 10 grams, 100 grams, 200 grams or smaller or intermediate or greater forces applied to the tip of the screw. In an exemplary embodiment of the invention, the strength of coupling is at least a factor of 1.2, 2, 4, 5, 10 of the weight of the screw.

In an exemplary embodiment of the invention, by coupling the screw to the screwdriver, a physician needs to hold only the screwdriver while aiming and inserting the screw into the body. This may be useful, for example, when the surgical incision and/or port being used is small.

In the example, of FIG. 1C, the circular recesses 512 at the screw head 504 have a tapering conical configuration. The compatible protrusions 514 at the screwdriver 510 have a cylindrical shape. Insertion of the cylindrical protrusions 514 into the conical recesses 512 can result in an assembly sufficient stable to enable said self-retaining feature. Easy and comfort handling of screws can be especially important when screws of small dimensions are involved (e.g., for treating small bones, such as in the case of distal radius plating).

Reverse tapering or hourglass tapering (wide entry, narrowing and then widening again) may be used in some embodiments of the invention. In such example, engagement is not only by friction due to compression, but also due to mechanical interference between a wider part of the blade and a narrower part of the receptacle (or vice versa). This may be useful, for example, when mounting of the screw can be done outside the body, so more force for mounting may be applied, but once in the body various forces may try to remove the screw. Once screwed in, axial retraction of the screwdriver should be sufficient to disengage the screw therefrom.

FIG. 1D shows a tapering example where cylindrical recesses 516 are provided at the screw head 504 and conical protrusions 518 are provided on screwdriver 510.

Referring again to FIG. 1C, a reference 521 indicates the depth of a receptacle 512 and a reference 523 indicates the length of a blade/protrusion 514 of screwdriver to 510. In an exemplary embodiment of the invention, there is a match between these two lengths. Optionally or alternatively, at least for one of receptacles, 521 is greater, to ensure good contact between the surface of the screw head and the surface of the screwdriver (e.g., between the blades). Optionally or alternatively, at least for one of the receptacles, 523 is greater, which may ensure that a maximum surface area of the receptacle is engaged. Such design variations may be used to deal with manufacturing variations.

Also shown in FIG. 1C is a virtual marker 525 which indicates the extension of cylindrical screw body. As shown, a receptacle 512 may straddle this line, to various percentages (e.g., 30%, 50%, 60%, 70% or greater or intermediate percentages of radial dimension of receptacle being not over the body). In other embodiments, the receptacle 512 is wholly on one or the other side of marker 525.

### Exemplary Guide Design

In an exemplary embodiment of the invention, screw head 502 includes one or more aiming guides for the screwdriver. Such guides may be useful for example, due to the greater difficult in screw engagement when the blade design is not axial and/or due to frictional or interference engagement between the screw and the screwdriver. In an exemplary embodiment of the invention, such guides are rotationally symmetric, so once the screwdriver engages the guide, the screwdriver can be rotated until the blades find the receptacles. Optionally or alternatively, the guide serves to transfer transaxial forces between the screwdriver and the screw.

FIG. 1E illustrates a head portion 800 of composite material bone screw or peg. Screw head 800 can be similar in various features to screw head 502 of FIG. 1A (e.g., it may be threaded (e.g., 802) and/or include, for example, four round recesses 804, arranged in a symmetric manner not in the center of the screw head but rather more peripherally.

In an exemplary embodiment of the invention, a protrusion the screwdriver (not shown in the Figure) to be connected to the screw head comprises protrusions matching the screw head recesses 804.

FIG. 1E shows two different geometrical mechanism which may be used separately and/or together to facilitate alignment of the screwdriver and the screw. Enabling fast, easy and precise connection between the screwdriver and screw can be to especially important when screws of small dimensions are involved and the implants and instrumentation have small dimensions (e.g., for treating small bones, such as in the case of distal radius plating).

In an exemplary embodiment of the invention, the top surface of screw head 800 defines an intermediate section 806 which is slightly sunken relative to the other surface areas (i.e., a screw head circumferential section 808 and an optional screw head central portion 810). Section 806 may have a ring shape, thus forming a small “tunnel” in which the four recesses 804 are located. Optionally, the width of said tunnel 806 is equal or slightly larger than the recesses diameter. The depth of this tunnel 806 may be in the range of, for example, 0.2 mm-0.4 mm Optionally, a protrusion 810 is provided in the center of the tunnel, and may extend higher than circumferential section 810. Optionally, the protrusion is provided as an alignment means instead of circumferential section 808.

Upon connecting the screwdriver to the screw head, the complementary protrusions in the screwdriver are guided into the tunnel 806 in the screw head top surface (e.g., the tunnel facilitates centralization of the screwdriver by receiving all of the protrusion of the screwdriver blade as a single element); an additional rotation of the screwdriver to either side can result in insertion of the screwdriver protrusions into the matching screw head recesses, to establish a proper engagement.

### Alternative Screw Geometry Design

FIGS. 2A-2D show an alternative design geometry for the screw head and screwdriver, in accordance with exemplary embodiments of the invention.

FIG. 2A displays a bone screw implant 530. Except for the connection means shape, other features described with reference to FIGS. 1A-1E may be used here as well.

As shown in FIG. 2A and FIG. 2B (a top view of FIG. 2A), there are three (but can be any number of recesses equals to- or higher than one or two) arcuate (e.g., banana-shaped) recesses 536 in the top surface of the screw head 532, which are optionally symmetrically arranged peripherally.

FIG. 2B shows a central point 537 of screw head 532, showing a first distance 543 along a maximal radius thereof that is larger than a second distance 545 within to which second distance protrusions 536 and/or a major part thereof are located. Also shown are a radial extent 541 and a circumferential extent (e.g., curved) 539 of a receptacle 536. As can be seen, circumferential extent 539 is greater than radial extent 541, for example, by a factor of, for example, 1.2, 1.8, 2, 2,5, 3 or greater or intermediate factors. In some embodiments, extent 539 is somewhat smaller than extent 541, for example, being 70%, 80%, 90% or intermediate or greater percentages thereof.

Referring now to FIG. 2C, in which the proximal portion of a bone screw 530 and the distal portion of a screwdriver 534 are shown. In an exemplary embodiment of the invention, screwdriver 534 comprises at its distal end three complementary elements 538, to engage with the screw head recesses 536. FIG. 2D illustrates a longitudinal cross section of FIG. 2C. As can be seen, in an exemplary embodiment of the invention, protrusions/blades 538 are curved.

FIG. 2E is a top view of a screw head illustrating various geometric considerations in accordance with exemplary embodiments of the invention.

Referring first to a receptacle 300, when in use, for clockwise turning, a force F will be applied to the receptacle, substantially only to its circumferentially leading face. The torque applied to the screw (for one receptacle) is M=F*R, where R is the average radius of receptacle 300. In an exemplary embodiment of the invention, the receptacle is shaped so that no forces are applied in direction other than perpendicular to the single circumferentially leading face. This may reduce local strains and/or damage. For example, the screwdriver blades and screw may be designed so that substantially all forces are applied to radial planes, e.g., planes that include the screw axis, so that less than 20%, 10% or intermediate percentages of the applied force are applied other than perpendicular to a radial direction.

As noted above, increasing R, allows a lower force F to achieve a same moment.

A further consideration is distribution of the force over a greater contact area, potentially reducing a local pressure. Thus, for example, increasing a width W of receptacle 300 or a depth D may reduce the pressure applied on an part of the screw and/or reduce local strains which may cause failure.

In an exemplary embodiment of the invention, the torque applied to drive a to screw is between 0.5 and 4 N*m and this figure (together with the failure points of the head material) is optionally used to design the screw geometry.

Reference 303 shows a receptacle with a narrowing 305, such that greater width is provided for contact areas at either side of the receptacle.

Reference 301 shows an example of a slot which is not radial, for example, being at an angle θ to a circumferential direction. Such a slot of length L has force F applied at an angle to its walls (and if the walls are long enough, possibly to an opposite wall as well). However, this may allow the force to be spread over a greater surface area.

Reference 307 is a receptacle which has one wider contact surface 302 (e.g., for unscrewing) and an inclined side 304. Force F is shown to be at an angle to inclined side 304.

Reference 310 shows a receptacle in which different force directions are provided at different parts thereof. For example, a less radially peripheral section 308 may be inclined, for example, to increase surface area and reduce local pressure, while a more radially peripheral portion 306 have substantially circumferentially perpendicular forces applied to it.

A screw in accordance with exemplary embodiments of the invention may have, for example, 1, 2, 3 or more receptacles of, for example, 1, 2, 3 or more designs such as described herein.

### Further Alternative Screw Geometry Design

FIGS. 3A-3D illustrate another alternative for interface between screw and screwdriver. FIG. 3A displays a bone screw implant 540. Except for the receptacle location, other features described for FIGS. 1 and 2 may be used here as well. As shown in FIGS. 3A and 3B (a top view of FIG. 3A), there are four (though a smaller or greater number may be provided) recesses 546 in the top surface of the screw head 542, which are optionally symmetrically arranged at the circumference of the screw head 542. This location of recesses may useful in cases where the risk of damaging soft tissue adjacent the screw head, by the thus defined sharp edges, is reduced.

Referring now to FIG. 3C, in which the proximal portion of a bone screw 540 and the distal portion of a screwdriver 544 are shown. In an exemplary embodiment of the invention, screwdriver 544 comprises at its distal end four complementary to elements 548 that engage the screw head recesses 546. FIG. 3D illustrates a longitudinal cross section of FIG. 3C.

### Another Alternative Screw Geometry Design

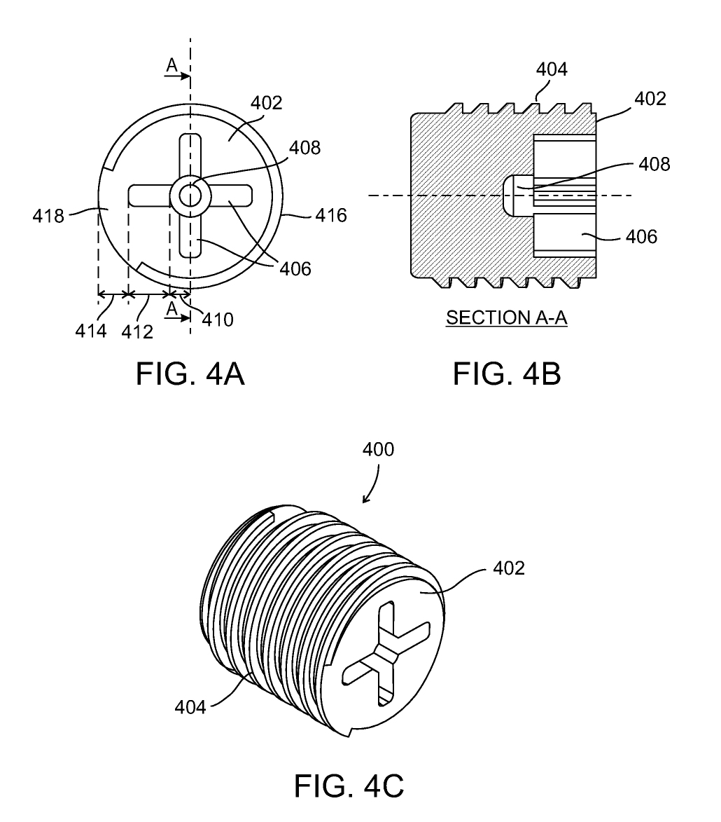

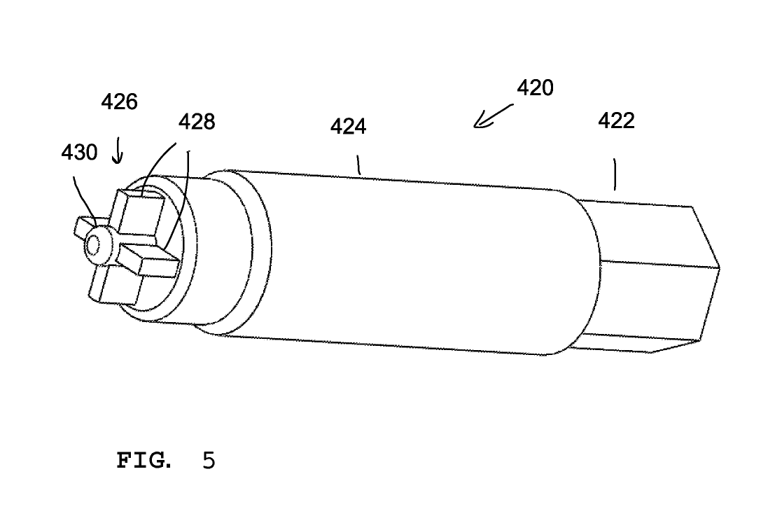

FIGS. 4A-4C illustrate an alternative geometry for a screw 400, in accordance with an exemplary embodiment of the invention.

FIG. 4A is a top view, FIG. 4B a side cross-sectional view and FIG. 4C a perspective view of screw 400. Screw 400 can include a head 402 and a body 404. Optionally, as shown, one or more slotted receptacles 406 are formed in head 402. Optionally, the slots meet, however, this is not required. In an exemplary embodiment of the invention, for example, as shown in FIG. 4B, the slots are deep, for example, 1 mm, 1.5 mm, 2 mm, 2.5 mm or intermediate or greater depths. In an exemplary embodiment of the invention, the screw has a same diameter head and body, allowing the slots to extend into the body 404 of the screw without substantial weakening thereof.

The design of screw 400 may be suitable for orthopedic uses where the screw does not contact bone, for example, for connecting and/or fixing in place spinal rods in spinal fixation systems.

In an exemplary embodiment of the invention, an optional central guide 408 in the shape of a cylindrical bore is provided along the axis of the screw. Such a guide may also act to functionally separate the slots and/or the slots parts to act as separate recesses.

In an exemplary embodiment of the invention, the working portion of a slot 406 is displaced at least a distance 410 form the axis of the screw and extends along a length 412 of the radius and stops a distance 414 from a periphery thereof. In an exemplary embodiment of the invention, distance 410 is between 5% and 30% of the maximal radius of screw head 402, length 412 is between 30% and 70% of said radius and/or distance 414 is between 5% and 30% of said radius.

Optionally or alternatively, screw head 402 is provided with an orientation guide, for example in the form of a depression 416 around some or all of its periphery and/or a protrusion 418 along some or all of its periphery.

While slots 406 are shown as being uniform, this need not be the case in all embodiments. In addition to tapering as described above for engaging the screwdriver, the depth and/or width of slots 406 may vary, for example, increase and/or decrease, as a function of the distance form guide 408.

FIG. 5 shows a design for an exemplary matching screwdriver 420. In an exemplary embodiment of the invention, screwdriver 420 has a shaft 424, a base 422 which may be a handle or be designed for engaging a handle or motor and a blade section 426. As shown, a cruciform blade is used, with two rectangular blade sections 428, to match slots 406 (e.g., as described with respect to FIGS. 1-3).

An optional protrusion 430 extends axially. In use, a protrusion 430 which is optionally tapered is inserted into one of slots 406 and/or guide 408. Once fully inserted, rotation of screwdriver shaft 424 will allow alignment of blades 428 and receptacles 406.

It is noted that other shapes and number of recesses at the head screw (and optionally complementary configurations in the screwdriver) may be provided in accordance with other embodiments of the invention, as well as combinations of such designs. In an exemplary embodiment of the invention, the shape of the receptacles is aligned with a fiber direction (e.g., circumferential) in the screw head, for example, providing curved receptacles.

### Exemplary Manufacturing Methods

Various methods for manufacture of the screw may be used. In an exemplary embodiment of the invention, molding of a composite material is performed to provide a shank with longitudinal fibers (e.g., that have high resistance to bending load), and a thread with fibers which were forced (e.g., using axial pressing) into the thread teeth during the molding process.

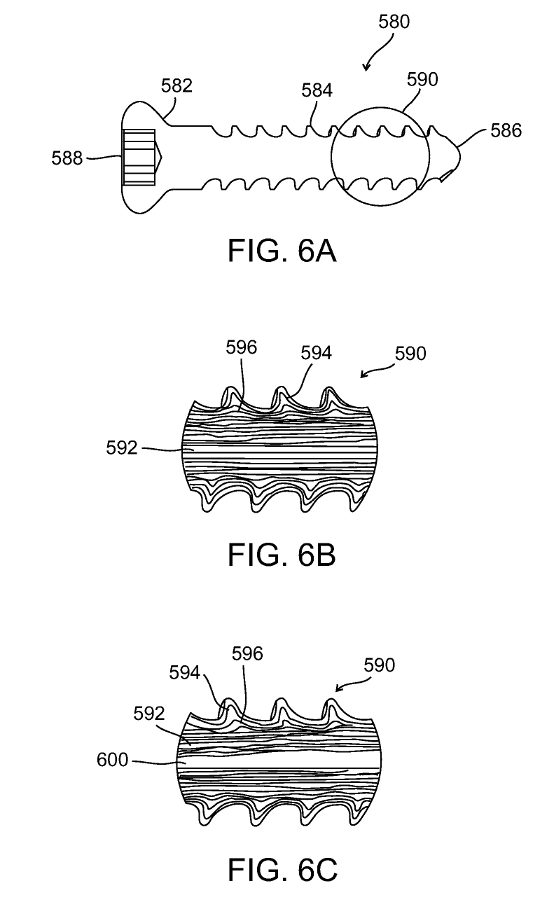

FIG. 6A illustrates a composite material bone screw 580, intended, for implantation, for example with intramedullary nails (including lag screws), bone plates or other implants, or intended for implantation as a stand-alone device. Various features may be provided, such as implant materials, implant radiopaque marker/s, implant dimensions, implant coating, self-tapping characteristics, connector to other instruments, etc.

In FIG. 6A, screw 580 has a head 582 and a threaded shank 584 and is intended for bone fixation, optionally being self-tapping and optionally including an insert at its distal end, embedded in the screw body, to protect the screw material against contact with bone, during driving of the screw, optionally as described in a related co-filed application.

Optionally, screw 580 tappers at its distal end 586, and/or includes connection means 588 to instruments such as a screwdriver at its proximal end. For example, any of the connection means/geometries described above may be used. However, it is noted that the manufacturing methods described herein may be used for manufacturing types of implants other than bone screws.

FIGS. 6B and 6C are magnifications of a portion 590 of screw thread 584 shown in FIG. 6A. Both figures illustrate potentially advantageous arrangement/configuration of the elongated reinforcing fibers within the composite material bone screw, in accordance with exemplary embodiments of the invention. As shown in FIG. 6B, a core 592 of the screw (e.g., except for the area of the thread and possibly some border areas) comprises straight longitudinal fibers, to provide for maximal resistance to bending loads. At a thread teeth region 594, the longitudinal fibers have a “wave” shape, as they were forced to wave into thread teeth 584. Such a thread may be beneficial for applications that require high pull-out forces (for example, in screws implanted with bone plates). At an intermediate area 596 between the core 592 and thread teeth region 594 the elongated fibers are optionally slightly waved. This screw design may contribute both to bending and pull-out resistance properties of a bone screw in accordance with some embodiments of the invention. In an exemplary embodiment of the invention, such a combination of fibers configurations may be produced from a composite material rod using a molding technique, where the core fiber elements are kept straight (tighten) out of the mold at a cold environment, and the circumference fiber elements are axially pressed, optionally by using a cylindrical shape press, so that they are forced to enter into the teeth-shape parts of the mold.

FIG. 6C illustrates a similar embodiment, however screw 590 comprises a cannulation 600 that enables its introduction over a K-wire.

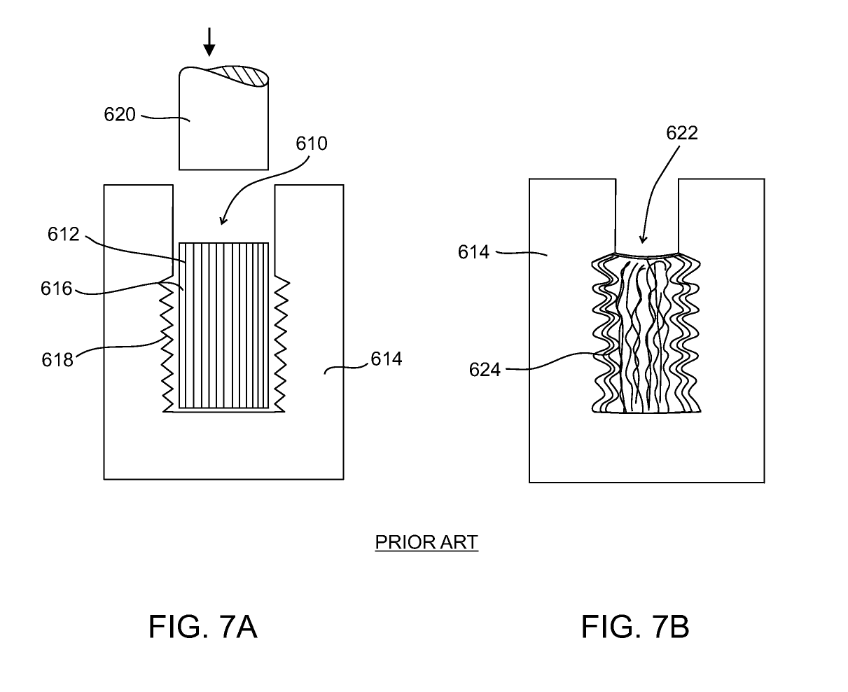

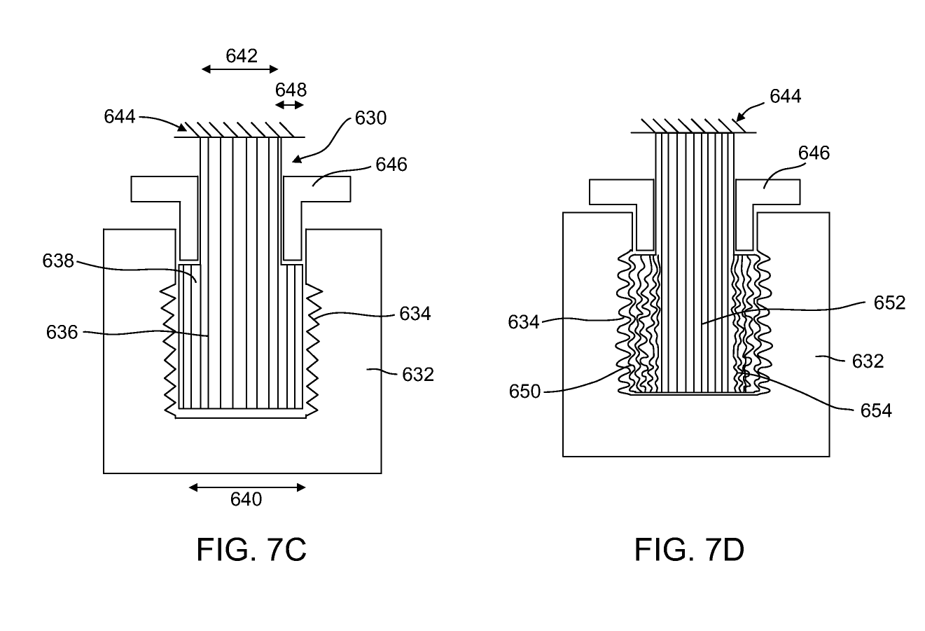

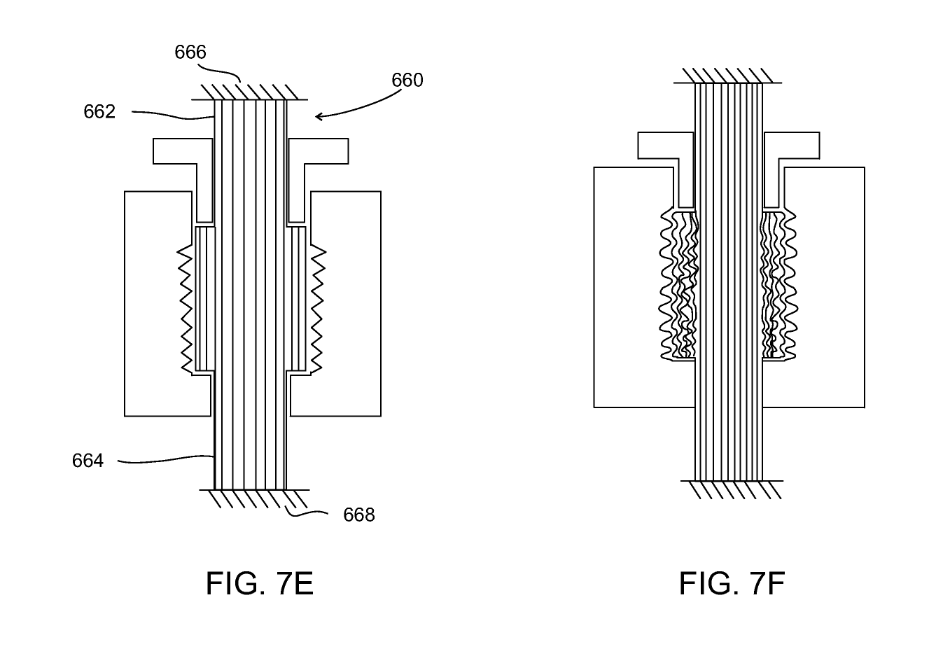

FIGS. 7A-7F schematically illustrate compression molding manufacturing methods of composite material bone screw, in accordance with some embodiments of the invention.

FIGS. 7A-7B show a prior art method as described in PCT publication WO 96/19336. FIG. 7A shows a composite material rod 610 prior to compression molding to process, comprising elongated reinforcing filaments 612 within polymer matrix 616. Rod 610 is placed within a mold 614, having at least along part of it a threaded (e.g., wave-like) configuration 618. During the compression molding process, a press 620 having the diameter of the rod 610 is used to axially press the rod 610 (under heat) into the mold. FIG. 7B schematically illustrates a fabricated thread/screw 622 following the process (as well as the mold 614). As shown in the figure, during the compression molding process the rod 610 is forced to gain the shape of the mold (e.g., the thread) and the longitudinal filaments become waved 624 not only in the threaded portion at the screw circumference but all over the screw, including its core.

In an exemplary embodiment of the invention, this method is used to manufacture screws. In an exemplary embodiment of the invention, at least the head portion is formed of unordered short chopped fibers, for example, 60% carbon and 40% PEEK, which has been found to be more resistant to compression stress. Other methods of manufacture using short chopped fibers may be used as well.

FIGS. 7C-7F schematically illustrate compression molding manufacturing methods of composite material bone screw according to some embodiments of the present invention.

FIG. 7C describes a composite material construct 630 before the molding process. Construct 630 is placed within a mold 632 with a threaded configuration 634 at least along part of the mold 632. Composite material construct 630 comprises elongated reinforcing filaments 636 within a polymer matrix 638. In an exemplary embodiment of the invention, construct 630 has a first, larger diameter 640 along its portion within the mold 632, and a second, smaller diameter 642 along its portion situated outside the mold. The rod outside the mold 632 is held 644 by restraining means, optionally at a cool environment. A press 646 optionally has a sleeve configuration, with thickness 648 optionally complying with the difference in rod diameters. Press 464 and/or mold 632 are optionally heated during compression.

FIG. 7D shows the construct 630 following compression molding (still within the mold 632). In an exemplary embodiment of the invention, because during the molding process the construct 630 is pressed by the sleeve press 646 only at its circumference while the core of the construct is restrained from folding by restrainer 644 (e.g., a circumferential rod holder), only the elongated filaments at the construct to circumference 650 are forced into the thread shape mold 634 and optionally made wavy. In an exemplary embodiment of the invention, the elongated filaments along most of the construct (e.g., its core) 652 remain straight. At intermediate locations, slightly folded elongated filaments 654 are optionally generated.

FIGS. 7E-7F describe a similar embodiment except that composite material construct 660 is held from both ends 666 and 668 and may include two smaller diameter sections 662 and 664. By keeping ends 666 and 668 in low temperature it is possible to hold the fibers in the screw core straight, during molding of the thread.

In an exemplary embodiment of the invention, the straight fibers are provided in a section having a diameter of between 50% and 80% or 100% of a diameter of the shaft of the screw (e.g., not including the thread).

### Additional Exemplary Screw/Peg Design

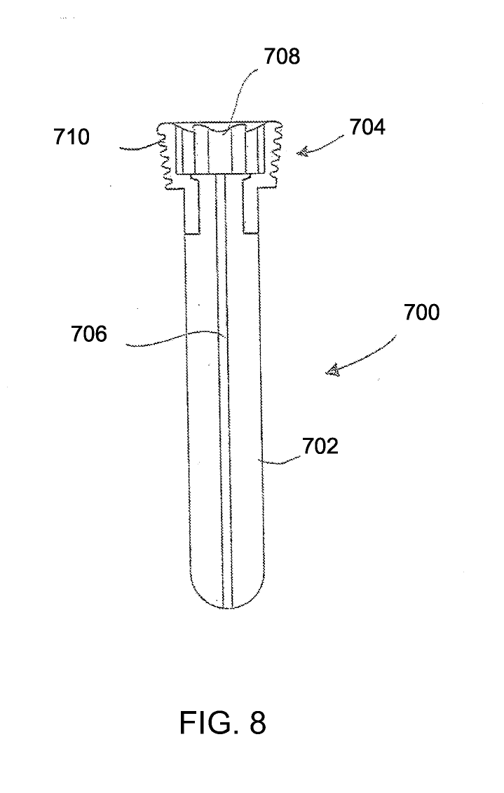

Reference is now made to FIG. 8, which schematically illustrates a composite material bone screw 700, comprising a shank 702 and a head 704 which is made of a different material, in accordance with an exemplary embodiment of the invention. Various features which were described herein are applicable here as well, including but not limited to implant materials, implant radiopaque marker/s, implant dimensions, implant coating, self-tapping characteristics, connector to other instruments, etc.

Screw shank 702 may be smooth (as shown in the figure), threaded or partially threaded. The composite material component may be visualized under fluoroscopy using radiopaque marker/s, for example a marker 706 along the center of the shank 702. At its proximal end, screw 700 comprises connection means 708 to instrument such as a screwdriver. Screw head 704 may be threaded 710, in order to enable locking of the screw 700 to another implant, such as a plate. However, screw head 704 with smooth circumference (i.e., a non-locking screw), is applicable as well.

In an exemplary embodiment of the invention, a resistance to compression pressure which is applied upon insertion (threading) and/or removal (unthreading) of the screw is provided by screw head 704 being made of material such as metal, optionally titanium alloy. Metal screw head 704 may be connected to the composite material screw shank 702 by various means, for example, including but not limited to geometric connection and/or adhesion means and/or mechanical connection and/or to using a compression molding process.

It should be noted that using a radially peripheral attachment mechanism allows a cannulation to be provided in the screw, optionally matching such a cannulation in a screwdriver, all potentially without interfering with the screwdriver blade geometry.

### Exemplary Materials and Manufacturing Methods

In an exemplary embodiment of the invention, the screw is formed of substantially linearly extending long reinforcing filaments in a polymer matrix (such as, but not limited to, polyetherketoneketone (PEKK), polyetheretherketone (PEEK), or other polyketone based polymers). Optionally, the reinforcing filaments are made of carbon. Alternatively, other reinforcing material may be used instead or in addition.

Optionally, threads include fibers wound around a core of the screw, for example, at or about the thread angle.

In an exemplary embodiment of the invention, the matrix comprises, in addition to the polymer, chopped fibers of carbon or other reinforcing material. Optionally, the chopped fibers have different orientations in the matrix. Optionally, the chopped fibers are of various lengths. In an embodiment, adding chopped reinforcing fibers into the polymer matrix, which is the weakest element in the composite material, increase the construction bending performance. Optionally, adding the chopped fibers allows a reduction in the content of the longitudinal fibers and/or replaces them.

In some embodiments of the invention, the contents of the reinforcing elements within the composite material is increased, in order to strengthen the material. In an exemplary embodiment of the invention, carbon fiber reinforced polymer (such as PEEK or PEKK) is used for a bone implant. In an embodiment, the carbon fibers volume content is about 60%. In an embodiment, the carbon fibers volume content is about 70%, optionally 80% or higher. In an embodiment, the prepreg tapes of carbon fiber reinforced polymer are produced with carbon fibers contents higher than 65%. Additionally and/or alternatively, the prepreg tapes are produced with carbon fiber contents of, for example, approximately 60%, and then later, part of the polymer is extracted outside from the tapes, optionally using high pressure and temperature. The carbon fibers can be for example IM7 or IM10 to manufactured by Hexcel Inc. or similar fibers.

In an exemplary embodiment of the invention, the implant is manufactured using compression molding process. In an embodiment, in order to strengthen the implant, the process of compression molding is performed under high pressure. In an exemplary embodiment of the invention, a bone implant is produced in compression molding from tapes of carbon fiber reinforced polymer (such as PEEK or PEKK), under pressure higher than 100 Atm., optionally higher than 400 Atm., optionally higher than 700 Atm., optionally higher than 1,000 Atm.

In an exemplary embodiment of the invention, a bone screw implant is formed from a composite material, such as carbon fiber reinforced PEEK or PEKK. In an embodiment, using compression molding, a rod is produced from prepreg tapes of longitudinal reinforcing fibers within a polymer matrix. The rod is then machined, to create the desired thread of the screw.

In another embodiment, the screw, including its thread, is manufactured from prepreg tapes of fiber reinforced polymer in compression molding process. During said process, the material is axially pressed under heat and pressure, so that folds are created in the elongate filaments and the material is forced to gain the shape of the thread at the mold circumference.

In another embodiment of the invention, the screw comprises a longitudinal core of, for example, carbon fiber reinforced polymer, and further comprises a profile winding, for instance with triangle cross section, that creates the thread around the said core.

In some embodiments of the invention, a composite screw is substantially radiolucent and is marked with radiopaque material, such as tantalum, to enable its visualization under imaging (e.g., fluoroscopy). Optionally, a radiopaque longitudinal thread is incorporated along the long axis of the screw. Alternatively and/or additionally, the marker is positioned at one or both ends of the screw, and/or at any location along the screw. Optionally, the marker has a shape of a dot, a ring, a pin, or other shape. Optionally, the screw comprises more than one marker, having the same or different shape and/or size.

### General

It is expected that during the life of a patent maturing from this application many relevant composite materials will be developed and the scope of the terms polymer, reinforcing fiber and composite material are intended to include all such new technologies a priori.

As used herein the term “about” refers to ±10%.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated to numbers and all the fractional and integral numerals therebetween.

### General

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

What is claimed is:

1. An orthopedic screw formed at least in part of a composite or polymer material, comprising:
(a) a generally cylindrical body defining an axis and including at least a partial threading thereon;
(b) a tip at a distal side of the body;
(c) a head at a proximal side of the body, said head having a maximal radius and defining at least one receptacle in the form of a recess, for a blade of a screwdriver,
wherein said recess includes a load bearing section adapted to engage said blade and which is closer to a circumference of said screw head than to an axis of said body.

2. A screw according to claim 1, wherein said screw is at least 90% by volume formed of a composite material or polymer.

3. A screw according to claim 1, wherein said load bearing section is within 40% of said maximal radius from a circumference of said screw head.

4. A screw according to claim 1, wherein said load bearing section is within 30% of said maximal radius from a circumference of said screw head.

5. A screw according to claim 1, wherein all of the load bearing sections of said screw head are at least 20% of said maximal radius distanced from said axis.

6. A screw according to claim 1, wherein at least 50% of the load bearing sections of said screw head are at least 40% of said maximal radius distanced from said axis.

7. A screw according to claim 1, comprising a plurality of recesses which are not connected.

8. A screw according to claim 7, comprising at least 3 unconnected recesses.

9. A screw according to claim 7, wherein a shape of said recesses is substantially circular.

10. A screw according to claim 7, wherein at least one of said recesses extends more in a circumferential direction than a radial direction of said head.

11. A screw according to claim 7, wherein said recesses are surrounded on all sides by a surface of said head.

12. A screw according to claim 1, wherein said recess forms an edge of said screw head.

13. A screw according to claim 1, wherein said recess is in the form of a slot.

14. A screw according to claim 1, wherein said recess is at least 1 mm deep at its most shallow portion.

15. A screw according to claim 1, wherein said recess has a varying width as a function of depth.

16. A screw according to claim 1, wherein said recess has associated therewith at least one insert formed of a material harder than said composite material or polymer, located at a load bearing surface thereof which is designed to receive force from said blade when said screw is driven.

17. A screw according to claim 1, comprising at least one screwdriver guiding geometry formed in said head.

18. A screw according to claim 17, wherein said geometry comprises a protrusion from a surface of said screw head.

19. A screw according to claim 17, wherein said geometry comprises a depression in a surface of said screw head.

20. A screw according to claim 17, wherein said geometry is rotationally symmetric with respect to said screw axis.

21. A screw according to claim 1, comprising at least one orientation guide formed along a circumference of said screw head.

22. A screw according to claim 1, wherein at least said head is formed of a composite material including as tensile elements short chopped fibers.

23. A screw according to claim 1, wherein at least said head is formed of a composite material having a better compression resistance than said body of said screw.

24. A screw according to claim 1, wherein said screw is in the shape of a lag screw.

25. A screw according to claim 1, wherein said screw is in the shape of a self tapping screw.

26. A screw according to claim 25, wherein said screw tip is configured to drill into bone.

27. A screw according to claim 1 provided in kit form with a matching screwdriver having a blade adapted to frictionally engage said at least one receptacle.

28. A screw according to claim 1 provided in kit form with a matching screwdriver having a blade adapted to engage said at least one receptacle.

29. A screw according to claim 28, wherein said kit comprises a plurality of bone screws.

30. A screw according to claim 28, wherein said kit comprises one of a bone nail and a bone plate.

31. A screwdriver for a composite orthopedic screw comprising a shaft and a blade section, wherein said blade section defines one or both of an axial hollow extending to a distal tip thereof and axially extending guide which is rotationally symmetric.

32. A kit comprising a screwdriver having a blade and an orthopedic screw having a receptacle for said blade, wherein a geometry of said blade interferes with a geometry of said receptacle, so as to provide friction engagement of said blade by said screw, when said blade is inserted into said receptacle.

33. An orthopedic screw having a generally cylindrical body having an axis and a thread on said body, wherein said body comprises a cylindrical section with a diameter of at least 50% of a diameter of said body, in which all elongate fibers are substantially aligned with said axis.
